(19) United States
(12) Patent Application Publication
(10) Pub. No.: US 2003/0092034 A1
(43) Pub. Date: May 15, 2003

(54) Analytical chip

(76) Inventors: Jonathan Cooper, Glasgow (GB); Vincent Benoit, Saint Louis (FR); James Stewart Aitchison, Toronto (CA)

Correspondence Address: MICHAEL BEST & FRIEDRICH, LLP, ONE SOUTH PINCKNEY STREET, P O BOX 1806, MADISON, WI, 53701

(21) Appl. No.: 10/181,093

(22) PCT Filed: Jan. 12, 2001
(86) PCT No.: PCT/GB01/00132

(30) Foreign Application Priority Data
Jan. 14, 2000 (GB) 0000896.1

(51) Int. Cl. C12Q 1/68; C12M 1/34

(52) U.S. Cl. 435/6; 435/287.2

(57) ABSTRACT

This invention relates to analytical “chips” known as biochips which are integrated microstructure devices able to perform biological or chemical measurements. In particular, the invention relates to an analytical chip comprising a substrate having an array of wells arranged on the substrate for receiving a fluid and at least one waveguide positioned transversely to the wells for receiving light from said wells in response to incident light into said wells. The invention is also concerned with a method of making the analytical chips and a point-of-care system for detecting biological or non-biological molecules.

This invention relates to analytical “chips”, so called integrated microstructural devices able to perform biological or chemical measurements. More particularly, though not exclusively, this invention relates to analytical chips which are used in optical assay systems.

Interest in the miniaturisation of optical, electro-chemical and di-electric sensor technologies has recently focused on the needs to perform biological assays in low volume, as might be appropriate for single-cell measurements, lab-on-a-chip technologies or genomics. A reduction in the size of the analytical structures offers many advantages over traditional technologies including low sample consumption, cheap unit prices and a number of distinctive functional properties inherent to the dimension of the devices (i.e. improved signal-to-noise ratios, laminar flow in capillaries, low diffusion times in microchambers and faster separation in the chromatographic micro-columns). The technology also provides the opportunity to produce highly parallel assay systems appropriate for high-throughput screening. For example, in determining ligand binding, assays involving antigen-antibody, antibody-antibody, protein-protein or DNA/RNA based assays may be used. In particular, these may be DNA-DNA, DNA-RNA, RNA-RNA or RNA-DNA or protein-protein.

As a consequence, established microfabrication techniques such as photolithography, reactive ion etching and silicon micromachining, initially developed for microelectronics and telecommunications, are now being adapted to the concept of lab-on-a-chip. The integration of microstructures with various functionalities such as micro total analytical systems (μTAS) and smart planar optical transducers (SPOT) has enabled analytical systems to be developed on-chip which include, for example, sample preparation, separation and detection.

Previous attempts to make lab-on-a-chip devices have used polymer layers such as polydimethylsiloxane (PDMS) sandwiched together. However, this method has the disadvantage that some of the polymers used may autofluoresce or adsorb light at some wavelength making them unsuitable for optical measurements. With these polymer structures, it may also be difficult to implement microfluidic methods including electro-osmosis due to problems in creating the correct surface chemistry. For example, it may not be possible to control the charge in the channels and/or the hydrophilic nature of the surface.

It is an object of at least one aspect of the present invention to obviate/mitigate one or more of the aforementioned disadvantages.

According to a first aspect of the present invention there is provided an analytical chip comprising a substrate having an array of wells arranged on the substrate for receiving a fluid and at least one waveguide positioned transversely to the wells for receiving light from said wells in response to incident light into said wells.

Preferably, said fluid is a liquid. Alternatively, said fluid is a gas.

Preferably, the wells are blind wherein fluid does not flow through said wells.

Alternatively, the wells extend through said substrate to allow fluid to flow through.

It is further preferred that the fluid is recirculated through said wells.

Preferably, the analytical chip is used for biological measurements and is known as a “biochip”.

Preferably, the biochip is about 1×2.5 cm.

Preferably, the substrate is a silicon, silica or glass wafer about 500 μm thick with about a 10 μm thick layer of thermally grown SiO₂ on the surface.

It is preferred that the wells are rectangular-shaped of about 50 μm wide and 50 μm deep.

Preferably, the biochip comprises a measurement chamber of about 200 μm×100 μm×50 μm yielding a structure of 1 nl.

Preferably, the wells have one input and one output.

Alternatively, the wells have one input and a plurality of outputs.

Preferably, there is a plurality of waveguides.

It is further preferred that the waveguides are about 9 μm deep.

Preferably, the waveguide channels are arranged to improve the efficiency of light collection.

Preferably, the waveguides are disposed orthogonally to the wells. It is further preferred that the waveguide have a width varying between 9 μm and 15 μm.

Preferably, the orthogonal waveguides are taper-shaped and have a starting width equal to the length of the measurement chamber, for example, 200 μm or 500 μm, and have a final width of 90 μm.

Preferably, on the inner surface of the wells a biological molecule which can bind a ligand is attached. It is preferred that the biological molecule has the ability to bind to a second biological molecule which contains a fluorophore group or causes a change in the optical property of the structure. Alternatively, the surface is functionalised by a biological molecule containing a fluorophore whose optical properties are changed on binding. Furthermore, it is preferred if the fluorophore groups are selected from those normally used in bio-analytical applications, for example, rhodamine and its derivatives, cyanine and its derivatives, Texas Red, proteins which contain fluorophores, natural and synthetic fluorophores, and tyrosine containing proteins.

According to a second aspect of the present invention, there is provided a method of making an analytical chip using a flame hydrolysis deposition process comprising:

hydrolysing halides in an oxy-hydrogen flame to form a low-density oxide soot;

depositing the soot on a layer of silicon glass;

sintering the soot to form a amorphous glass;

etching the amorphous glass to form a plurality of waveguides;

depositing a further layer of amorphous glass by flame hydrolysis deposition to act as a cladding layer to the waveguide; and

then performing a further etching process on the cladding layer to form an array of flow channels.

Preferably, the soot is deposited by using an aerosol spray.

Preferably, the analytical chip is a biochip.

Preferably, the halides are metal halides. More preferably, the halides are SiCl₄, GeCl₄, BCl₃, and POCl₃.

Preferably, the halides are in different feedlines to enable sequential deposition or co-deposition.

Preferably, the soot forming the waveguide layer is sintered at 1350° C. for 2 hours forming a layer of 9 μm.

Preferably, a mask is used in the second flame hydrolysis deposition layer during the etching process.

It is further preferred if the final device is sintered at 1100° C. for 2 hours.

Preferably, on the surface of the flow channels, biological molecules or fluorescently labelled biomolecules are added by injecting an immobilisation solution of saturated primer solution through the flow channel.

It is further preferred if the primer solution is a functionalised silane.

Preferably, the above procedure is repeated to provide a multi-biochip structure. Alternatively, a multi-layer structure is obtained by forming a biochip with a waveguide and flow channel layer and then using anodic bonding to bond said biochip onto another biochip.

Preferably, the anodic bonding method comprises disposing a thin layer of Ti or Ni between the surfaces of two adjacent biochips.

In a further alternative arrangement of providing a multi-layer biochip, an adhesive layer (e.g. a polymer or a glass) is used to attach two single layer biochips.

According to a third aspect of the present invention, there is provided apparatus for fluorescence measurements comprising:

a light source for irradiating an analytical chip in a first direction with incident radiation, a light detection system for collecting emerging light from the analytical chip said emerging light being in a direction substantially in-line to said first direction.

According to a fourth aspect of the present invention, there is provided apparatus for fluorescence measurements comprising:

a light source for irradiating an analytical chip in a first direction with incident radiation, a light detection system for collecting emerging light from the biochip, said emerging light being in a direction substantially orthogonal to said first direction.

Preferably, the analytical chip is a biochip.

Preferably, the light source is a HeNe laser.

Preferably, the detection system is a CCD.

According to a fifth aspect of the present invention, there is provided an integrated analytical chip comprising a light source for irradiating an analytical chip in a first direction with incident radiation, a light detection system for collecting emerging light from the analytical chip, said emerging light being in a direction substantially orthogonal to said first direction.

According to a sixth aspect of the present invention, there is provided an integrated analytical chip comprising a light source for irradiating an analytical chip in a first direction with incident radiation, the light detection system for collecting emerging light from the analytical chip, said emerging light being in a direction substantially in-line to said first direction.

According to a seventh aspect of the present invention there is provided a point-of-care system for detecting a biological or non-biological molecule or component, said system comprising:

an analytical chip according to at least the first aspect of the invention;

a light source for irradiating the analytical chip with incident light;

a light sensor for detecting non-incident light from said chip, said non-incident light containing information about said biological or non-biological molecule or component;

signal processing means for processing said non-incident light and for extracting data representative of said biological or non-biological molecule or component and means for presenting the results of said detection.

Preferably, the means are displayed visually, transmitted audibly or transmitted remotely.

Conveniently, the molecules are those normally required for clinical measurement e.g. DNA, RNA, proteins, enzymes, antibodies of antigens.

Embodiments of the present invention will now be described, by way of example only, with reference to the accompanying drawings in which:

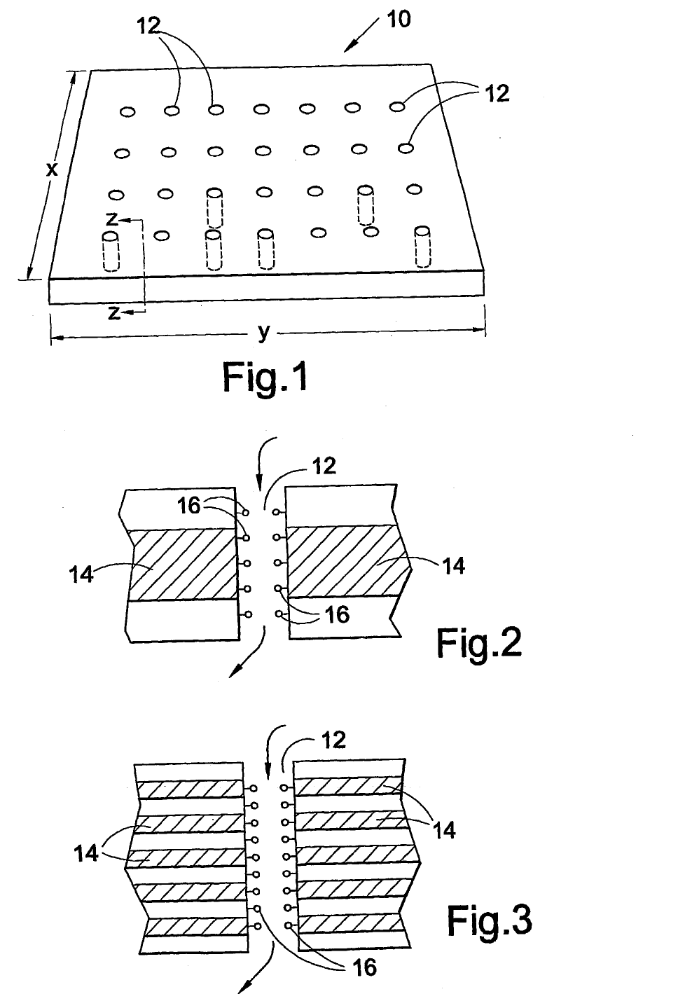

FIG. 1 is a perspective view of a biochip according to a first embodiment of the invention;

FIG. 2 is a sectional view of the line 2-2 of the biochip of FIG. 1;

FIG. 3 is a sectional view similar to FIG. 2 but of a multi-layer biochip;

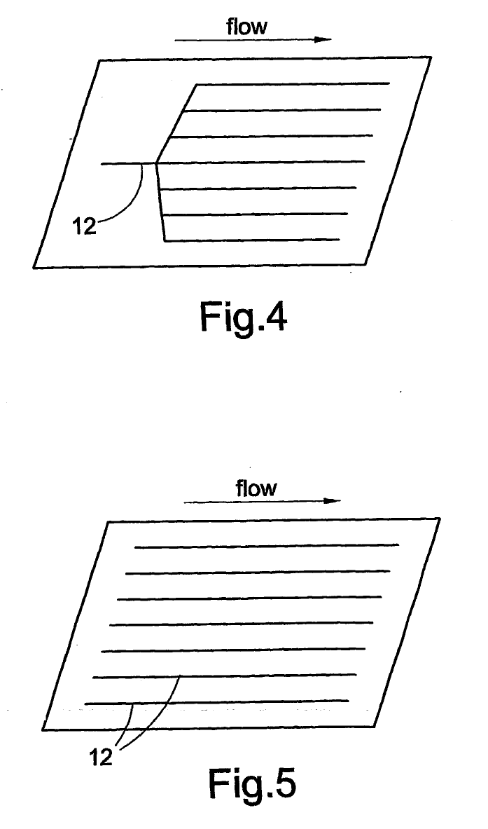

FIG. 4 is a diagrammative representation of a biochip with a single flow channel diverging into multiple flow channels;

FIG. 5 is a diagrammative representation of a biochip with parallel flow channels;

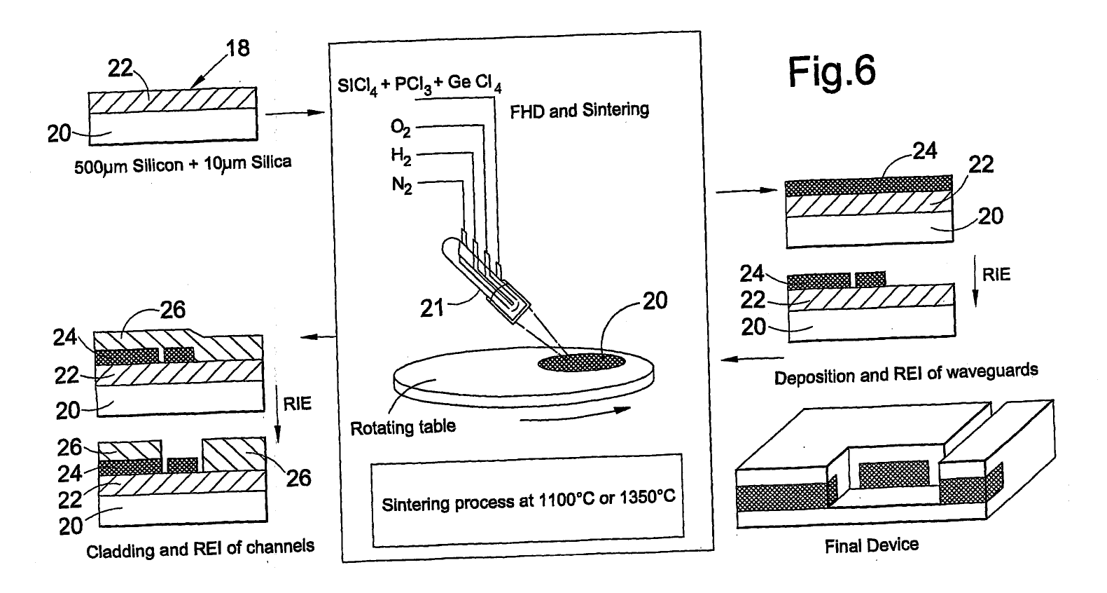

FIG. 6 represents schematically the steps involved in method of forming the biochip of FIG. 1;

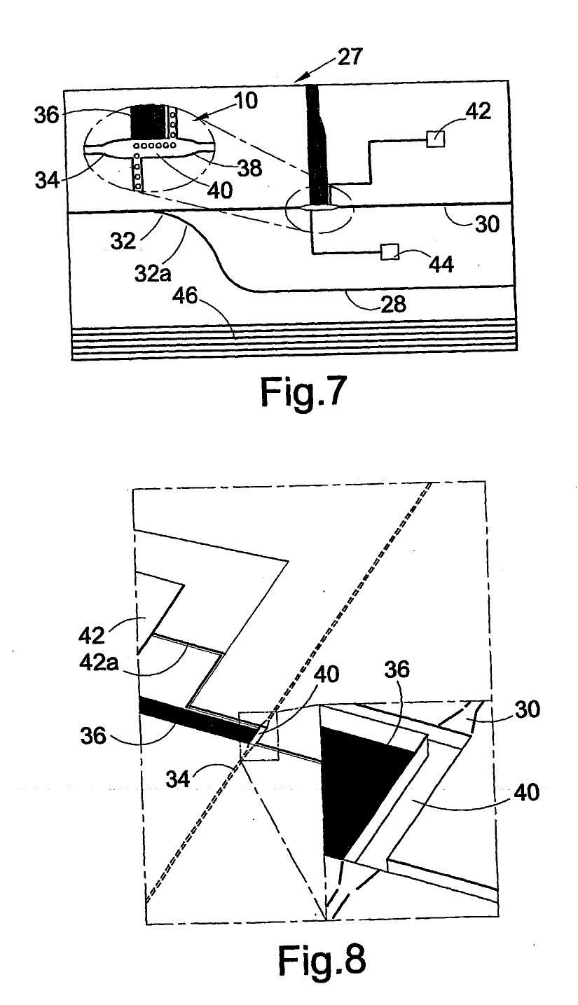

FIG. 7 is a diagrammatic plan view of an optical sensor using the biochip of FIG. 1;

FIG. 8 is a scanning electron microscope (SEM) image of part of the biochip surface of the optical sensor of FIG. 7;

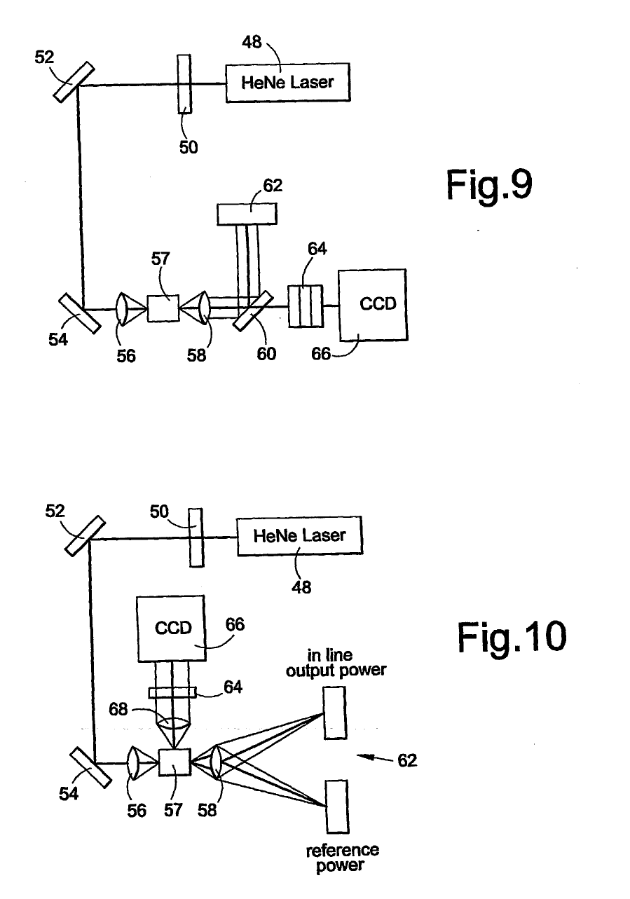

FIG. 9 is a schematic representation of apparatus for fluorescence measurements wherein a detector is located to detected in-line emergent light from a biochip;

FIG. 10 is a schematic representation of apparatus for fluorescence measurements wherein a detector is located to detect orthogonally emergent light from a biochip;

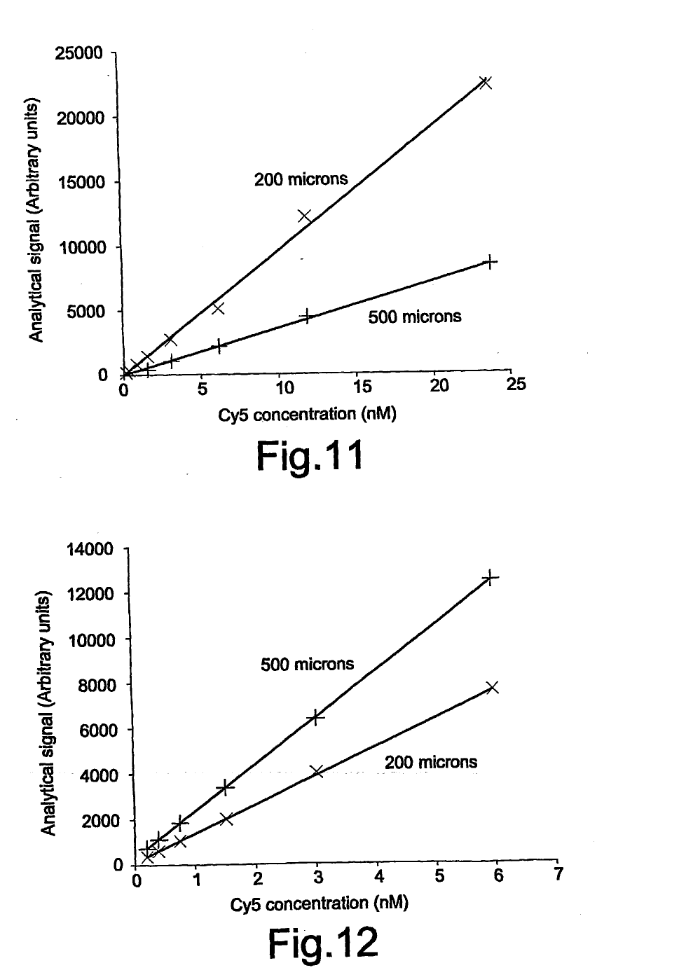

FIG. 11 is a calibration curve for the in-line format as shown in FIG. 9;

FIG. 12 is a calibration curve for the orthogonal format as shown in FIG. 10; and

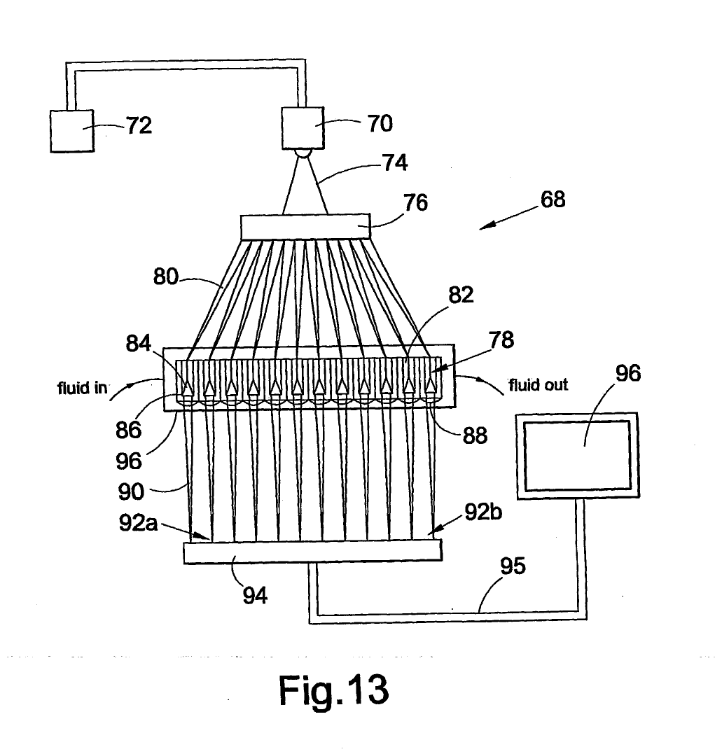

FIG. 13 shows a device for assaying samples using the multi-layer bio-chip of FIG. 3.

Referring to FIG. 1, there is illustrated a biochip 10 with a plurality of etched wells 12. The biochip 10 is rectangular in shape and is of about 1×2.5 cm. The wells 12 are cylindrical in shape and have a diameter of 10 μm and a depth of 300 μm. Furthermore, as shown in FIG. 1, the wells 12 are arranged in rows and columns with a total of 28 wells 12 in the biochip 10.

FIG. 2 is an enlarged sectional view along the lines 2-2 of FIG. 1. FIG. 2 shows a single-layer structure with a well 12 and single waveguide layer 14. The well 12 is cylindrical and is 10 μm wide and 300 μm deep. On the surface of the well 12, there is a plurality of cyanine 5 (Cy5) (Registered Trademark Amersham Pharmacia) fluorophores 16. The fluorophores 16 are attached by silane chemistry known to those skilled in the art.

In FIG. 3, there is shown a multi-layer construction which has five waveguide layers 14, each of which has a thickness of 10 μm separated by 40 μm layers of polymer or glass. The well 12 is cylindrical and is 10 μm wide and 300 μm deep. On the surface of the well 12, there is a plurality of Cy5 fluorophores.

In FIGS. 2 and 3 fluid is shown entering at the top and exiting at the bottom through the well 12. The fluid simply passes through or is recycled using a fluid circuit (not shown in the interests of clarity).

FIGS. 4 and 5 are representations of different types of fluid supply channels 15. FIG. 5 shows a number of parallel fluid supply channels 15. FIG. 4 shows an alternative arrangement in which one channel diverges to many supply channels.

FIG. 6 shows schematically the steps in the method of forming the waveguides 14 and etched fluid supply channels 15. Firstly, a substrate 18 consisting of a three inch diameter silicon wafer 20, approximately 500 μm thick, with a 10 μm thick layer of thermally grown SiO₂ 22 on the surface of wafer 20 is used as a substrate 20. Planar high silica content films are then deposited on the SiO₂ layer 22 by flame hydrolysis deposition (FHD). FHD involves the hydrolysis of gaseous metal halides, such as SiCl₄, GeCl₄ and BCl₃ in an oxy-hydrogen flame to form low density oxide soot.

Each of the gaseous metal halides are carried in a separate feedline so that different planar glass material systems are produced by either sequential deposition or co-deposition of the appropriate soots. A flow of nitrogen is used to transfer the gaseous metal halides along a gas line. A flow valve is used to control the pressure in the gas line. Overall the hydrolysis process is described by the following reactions:

SiCl₄(g)+2H₂O(g)→SiO₂(s)+4HCl(g)

GeCl₄(g)+2H₂O(g)→GeO₂(s)+4HCl(g)

2BCl₃(g)+3H₂O(g)→B₂O₃(s)+6HCl(g)

2PCl₃(g)+3H₂O(g)+O₂(g)→P₂O₅(s)+6HCl(g)

In this example, SiO₂ and both B₂O₃ and GeO₂ glasses were co-deposited to form a composite low density oxide soot. The soot was then sintered at 1350° C. for 2 hours to form a glass layer 24 with a thickness of 9 μm. The glass layer 24 is used to form the waveguides 14. After formation of this initial layer of glass 24, the waveguides 14 are defined using a reactive ion etching process involving a bi-layer mass comprising Nichrome and photoresist. FHD was then used again to deposit a second glass layer 26 of a different composition to the first glass layer 24 and having a lower refractive index to act as the waveguide cladding layer with the refractive index matching that of the thermal oxide underlayer using phosphorous pentoxide (P₂O₅) and boron oxide (B₂O₃) doped glasses to a thickness of 30 μm. The whole device was finally sintered at 100° C. for 2 hours. A torch 21 is moved across the surface at a rate of 1 revolution per second to give uniform deposition as the glass substrate rotates.

The waveguides 14 are etched using any suitable known method for patterning of FHD silica-rich glasses creating channels with depths of up to 40 μm.

One reactive ion etching process uses a bi-layered reactive ion etching mask deposited on the device by photolithography. The bi-layered mask comprises a layer of NiCr (not shown in the interests of clarity) and which gave a very selective and high aspect ratio edge, but which was prone to alloy redeposition coated with a less selective AZ4562 photoresist. This outermost polymer layer, which is the upper layer of the bi-layer mask and also not shown in the interest of clarity, acts as a sacrificial layer that greatly reduces the rate and thus the amount of NiCr redeposition. The photolithography protocol used to create the mark is described as follows.

FHD coated substrates were cleaned in a Pirhana solution (90% H₂SO₄/10% H₂O₂) and a layer of Nichrome (60% Ni/40% Cr) 140 nm thick was deposited by thermal evaporation. A 6.2 nm thick layer of AZ4562 photoresist was then spin-deposited (4000 rpm, 30 seconds) and baked at 90° C. for 30 minutes. The resist was patterned by resist exposure through a ferric oxide photomask and was subsequently developed and post-baked at 120° C. for 20 minutes. The final pattern was transformed onto the NiCr mask using a wet edge of 0.6M acetic acid/0.37M ammonium hexanitrocerate. The thick photoresist layer was retained after the wet etch as this was found to prevent redeposition of the mask during dry etching. In this way, vertical channels of about 40 mm in depth are produced consistently. The masking procedure was performed twice, first to create the waveguides 14 and subsequently to produce the flow channels 12 later in the chip manufacture process.

An Oxford Plasma Technology RIE80 reactive ion etching machine was used which was cleaned prior to each etch run with an oxygen plasma. The substrates 18 coated with the polycrystalline layer 24 are coated with the bi-layered mask and are etched at rate of 94 nm min⁻¹ using CHF₃. The flow rate etching pressure and RF power were 25 sccm, 60 mT and 190 W respectively giving a selectivity of 5.5 to 1 over the photoresist layer. Deep silica etching of about 40 mm requires 6 to 7 hours. A DekTak surface profiler was used to measure the mask thickness prior to and after etching from which the values from the FHD glass and mask etch rates as well as the glass to mask seal activity were calculated. The surface profiler was also used to determine the roughness of the etched surfaces and the etched profiles of the samples were examined with a Hitatchi S-800 scanning electron microscope (SEM).

An anodic bonding technique was adapted by the addition of a titanium (Ti) layer in order to be able to seal the wells 12 etched in the flame hydrolysis deposited glass with a sodium-rich glass (Pyrex 7740, Corning). Once the waveguides 14 and fluid supply channels 15 are formed in the device, a mechanical polishing step is performed to yield a flat surface. Because electronic induction is required between the two surfaces to be bonded, a layer of Ti (75 nm thick) was deposited onto the whole surface. The Ti covering the sensing regions of the floor (floor and walls of the measurement chamber) is then selectively removed by photolithographic masking and a standard wet edge before bonding.

In FIG. 7 there is shown a diagrammatic plan view of an optical sensor which integrates microfluidics and optical waveguides, generally indicated by reference numeral 27, incorporating a biochip 10 shown in enlarged detail for the clarity in the enlarged area 29. A waveguide 28 is used for reference measurements of the excitation power. There is also an in-line output waveguide 30; a Y branch 32; a tapered input waveguide 34; an orthogonal output waveguide 36; an in-line tapered output waveguide 38; a measurement chamber 40; reservoirs 42, 44 and straight waveguides 46.

In addition to the waveguides 28, 30, 34, 36, 38, 40, 42, 46; the chip comprises a measurement chamber 40 connected to two reservoirs 42, 44 through microchannels 15 μm wide and 38 μm deep. The overall size of the device is about 1×2.5 cm. The measurement chamber 40 in FIG. 7 has dimensions of 200 μm×30 μm×38 μm, yielding a volume of 230 pl. The device features one input waveguide and two output waveguides such that both in-line and orthogonal configurations can be used. The orthogonal waveguides 36 have a starting width of 200 μm equal to the length of the measurement chamber 40 and a final width of 90 μm. In input waveguide 32 has a starting width of 9 μm diverging to 15 μm at the input to the measurement chamber 40. The output waveguide 30 has input width of 15 μm which converges to 9 μm.

As shown in FIG. 7, a set of straight waveguides 46 is fabricated within the optical device in order to measure the levels of both autofluorescence and losses within the flame hydrolysis deposited glass over the range of analytical wavelength to provide a reference level for the glass. In addition, an optical Y branch 32 featuring a narrow “neck” to minimise the variations of the splitting ratio versus the lateral deemed displacement is fabricated in order to enable reference measurements of the excitation power to be made. The radius of the bend 32a is 20 mm and the refractive index difference is 0.45% resulting in less than 0.04 dBcm⁻¹ losses in the bend.

In FIG. 8 there is shown a SEM image of the integrated device. The input waveguide 34 is shown as a dotted line and the output 36 is shown as a wider solid line. The measurement chamber 40 is shown in detail and is shown as being wider than the entering and leaving channels. a reservoir 42 for reagent storage is also seen on the left, with a short connecting channel 42a.

As shown in FIGS. 9 and 10, different optical configurations can be used; FIG. 9 shows a configuration where “in-line” waveguides collect light; and FIG. 10 shows a configuration of where “orthogonal” waveguides are used to collect light.

In FIG. 9, a laser line filter 50 is used to control the output of the HeNe laser 48. a succession of mirrors 52, 54 are then used to direct the laser beam through a lens 56 onto the optical sensor 27. a lens 58 is then used to collimate the light onto a dichroic mirror 60. Some of the collected light is then reflected orthogonally onto a silicone photodetector 62 with the rest of the light passing “in-line” through fluoroescence emission filter 64 onto a CCD 66.

The detector used was a highly sensitive black and white CCD camera (Spectra Video SV10K, Pixel Vision). The CCD 66 was cooled to 255K using the built-in Peltier water cooling system to minimise the dark current a PMT (Model H5700, Hamamatsu, Hamamatsu City, Japan) was also used to measure the autofluorescence of the flame hydrolysis deposited glasses. The optical filters 64 are from Omega Optical (Glenn Spectra, UK), and comprise a narrow-band excitation (633NB3.0) filter and several emission filters (670DF40 filters). For the in-line optical configuration, it is necessary to place three emission filters 64 between the optical sensor 27 and the CCD 66 to block the excitation radiation a dichroic mirror 60 (Omega Optical) was used to allow measurement of the excitation radiation exiting the output waveguide.

In FIG. 10, where like numerals refer to like parts as in FIG. 9, the optical layout from the laser 48 to the optical sensor 27 is identical but most light from the optical sensor 27 is reflected orthogonally to the incident light and passes through a lens 68, an emission filter 64 and a CCD 66. The rest of the light passes straight through the optical sensor 27 and then passes to a lens 58 from where it is directed onto a silicone photodetector 62. Two detectors are used so that a reference signal is detected from the waveguide 28 and from waveguide 30, the light passing through the fluid which has its refractive index changed is measured. In all the fluorescence measurements, the laser power is maintained at 2 mW.

In the orthogonal set-up only one filter 64 was required due to the greatly improved signal-to-noise ratio.

The excitation radiation was measured simultaneously from the reference waveguide of the Y junction 32 and from the in-line output waveguide 38.

Prior to use, the optical sensor 27 is cleaned in Pirhana solution. An addition to removing any adventitious dirt, this cleaning routine increased the hydrophilicity of the glass surface which facilitates the subsequent filling of the channels and the measurement chamber by capillary action. The blank (Bk) level was measured in each optical configuration by filling the measurement chamber 40 with water. The total signal (some of the blank signal) and the analytical signal (S) was then measured for increasing concentrations of the analyte. The dark (Dk) current and associated noise of the CCD camera were also measured under the conditions of the analysis.

The optical sensor 27 is also placed under an epifluorescence microscope (Nikon Nicrophot, Nikon, UK) so that fluorescence could be excited and collected directly within the channels and measurement chamber 40 from the top without using the waveguide. The optical sensor 27 was imaged with the CCD camera which was coupled to the photoport of the microscope to check that both the channels and the measurement chamber 40 can be filled and emptied uniformly in a reproducible fashion and that the washing procedure employed between measurements was satisfactory to ensure that no significant memory effects due to absorption of the sample molecules onto the walls of the channels and measurement chamber 40 took place.

Transmission losses were measured at approximately 0.5 dBcm⁻¹ at the wavelength of 633 nm used in this analysis. This level is higher than that observed at the longer telecommunication wavelength as a consequence of the increased level of Rayleigh scattering at shorter wavelengths. The power split ratio in the Y branch was measured to be 97:3, which was also in approximate agreement with the 90:10 ratio obtained by simulations carried out using computer software. The levels of power transmitted through the measurement chambers 40, the latter being filled with water, are measured at 21% for the 200 μm long measurement chamber. The level of autofluoroescence of the optical circuitry was characterised using the PMT. The transference curves of the PMT provided calibration of the radian sensitivity versus the output voltage. The level of autofluorescence of the FHD glasses at 633 nm was estimated to be as low as 0.150 pW cm⁻¹ for an excitation power of 500 mW.

FIGS. 11 and 12 show calibration curves collected for a model analytical system, measuring signal generated by the fluorophore Cy5. These compare the 200 μm width measurement chamber 40 with a measurement chamber of 500 μm width and the orthogonal configuration a high level of analytical signal was collected in a larger measurement chamber (570 pl) due to the fact that the cross-sectional area of the waveguide that interfaces with the measurement chamber was greater.

In the in-line configuration, the smaller (230 pl) measurement chamber (200 μm width) was characterised by approximately 3.5 increase in the level of blank signal as well as total signal as compared to the signals of the larger (570 pl) measurement chamber (500 μm in width). This is in compliance with the difference in the levels of the power transmitted through the measurement chambers and stems from the different extents of overlap of the excitation and collection volumes in the differently sized measurement chambers. Absorption effects are not significant over the range of Cy5 concentrations used as indicated by the absence of roll-over at the higher concentration.

Table 1 shown below shows a comparison of the analytical figures of merit for the different optical configurations used in fluorescence measurements with the same concentration of Cy5 and water (3 nM). As expected, for each measurement chamber size, the orthogonal response is characterised by a lower level of background and higher signal-to-noise (S/N) and signal-to-background (S/B) ratios. Accordingly the experimental detection limit (DL) was found to be lower in the orthogonal configuration and was measured as 20 pl equivalent to 10 zmol of material. (NB 1 zmol=1×10⁻²¹ mol).

TABLE 1

| | Op- | | | | | | |
|---|---|---|---|---|---|---|---|
| Chamber | tical con- | Dark | Blank | Total | | | DL |
| width | figuration | (Dk) | (Bk) | Signal | S/N | S/B_{[JR18]} | (pM) |
| 200 μm | In-line | 1100 | 7278 | 9804 | 16.84 | 0.41 | 188 |
| | Ortho- | 1100 | 1550 | 5500 | 26.33 | 8.78 | 95 |
| | gonal | | | | | | |
| 500 μm | In-Line | 1100 | 1930 | 2670 | 4.93 | 0.89 | 1500 |
| | Ortho- | 1100 | 1550 | 7840 | 41.93 | 13.98 | 20 |
| | gonal | | | | | | |

Fluorescence measurements were also made from aqueous solutions of the Cy5-labelled 53 mer oligonucleotide (ODN) were also carried out in the 570 pl measurement chamber in the orthogonal configuration. At the lowest concentration used, 1.25 nM the limiting noise was found to be that from the dark current. As expected, as the concentration of ODN was increased (up to 180 nM) the analytical noise was found to increase by more than two orders of magnitude owing to Rayleigh scattering of radiation by the ODN molecules.

FIG. 13 shows a detailed diagram of an assay apparatus 68 using an optical sensor 27 having a multi-layer biochip 10 as shown in FIG. 3. The apparatus 68 includes HeNe laser excitation radiation source 70 connected to a power supply 72. The HeNe laser emits radiation 74 at a wavelength of 632 nm with a beam diameter of 0.5 mm-5 mm. A diffraction grating is etched on a quartz plate 76 designed specifically for use with 632 nm light. It is a straight-forward matter for those of ordinary skill in the art to produce such diffraction gratings.

The quartz plate 76 diffracts the radiation 74 into a pattern of almost parallel incident beams 80, corresponding to the pattern of wells 14 in the biochip 10.

Each incident excitation beam 80 excites a sample 84 in each well 12, which thereby emits emission radiation. In this example, Cy5 is used as a fluorophore which absorbs light at 632 nm and emits light at 640 nm to 800 nm with a pH with local environment dependent peak about 670 nm. The re-emitted or emergent light from the fluorophore is directed by waveguides 14 in a direction orthogonal to the incident light towards CCD arrays 92a, 92b which detect the emergent light from the fluorophores.

The CCD detected signals are processed in a signal processor 94 and the processed signal 95 then displayed on a display screen 96.

Various modifications may be made to the optical sensor and analytical chips hereinbefore described without departing from the scope of the invention, for example, the wells may have a diameter in the range of 5-500 μm and a depth in the range of 10-1000 μm. The waveguides can be configured to generate an evanescent field which is used to excite a fluorophore. Also the input waveguide can be split to form an interferometer sensor, for example, a Mach-Zehnder interferometer wherein a binding array which occurs in one path is used to modify the refractive index of light on that path compared to a refractive path, leading to a phase change resulting in a change in transmission which is quantitatively related to the binding assay. This example does not require use of a fluorophore and can be used as a biological assay. A nickel layer may also be used in anodic bonding instead of titanium. The measurement chamber can be of any suitable width for example 500 μm.

A further modification is that the fluid may be recirculated to maximise the amount of binding of the biological or non-biological molecule or component on the well surface to maximise the response signal for the detection system. The fluid is recirculated by well-known microscopic fluidic techniques.

The biochip or analytical chip may be planar (2-D) or 3-D with fluidic channels, wells, pores or other functional structures such as reservoirs, capillaries, etc. integrated into the chip surface wherein thermal, optical or electrochemical signals may be detected. The optical measurements may be made in free space with lenses and detectors. One or more biomolecules may be spatially located in the chambers, channels or on the waveguides to create a biosensing structure which will bind a ligand such as an antibody, an antigen, DNA, RNA or an ion.

The biochip described herein may be used in hand-held medical devices, fluorescence measuring equipment, chemical and biological assay measurements, in-situ chemical measurements, screen assay systems and environmental sensing, e.g. using modulation of florescence from functional metal chelator, on bending of specific ions. In particular, the biochips may be used for the detection of fluorescently labelled antibodies and measuring the presence or absence of human chorionic gonadotropin (HCG) in urine as a pregnancy test. Alternatively, immunoassays may be carried out on blood samples for the detection of heart disease and AIDS.

A further alternative is that an integrated analytical chip may be used wherein a semiconductor laser is used as a light source for the biochip. The light is detected by a CCD, CMOS or a photomultiplier tube (PMT).

The biochip may also be used in a point-of-care system which may be a fixed or hand-held device. The point-of-care system may be used for screening transmitted diseases at a blood bank, detection of heart disease and analysis of genetic profiles in a neonatal unit to screen for possible diseases such as cystic fibrosis, PKU, Downs Syndrome etc.

Furthermore, the biochip may be used to detect the presence of genetically modified food.

The biochip may also be used as a chelator for detection of metals, such as Se, Cd, Hg, Ag and Ca. Detection of Ag is useful in photographic applications. Detection of the amounts of Ca in hard water is useful when calculating the amount of softener to add. The biochip may also be used to detect the presence of agrochemicals such as pesticides.

The biochip may also be incorporated with mobile phone technology so that data acquisition on the chip is interfaced with a mobile phone.

A yet further modification is that disposable cassettes may be used in the biochips. These operate using dry reagent chemistries.

A yet further modification is to modify the biochip so that gases may be passed through the wells instead of liquids. Suitable gases are, for example, hydrocarbons and fluorocarbons. For this type of analysis the biochip is modified by applying a polymer coating containing a fluorophore to the inner surface of the wells which have different abilities to partition or bind different gases and therefore are used to quantify or detect the presence of gases. Fluorophores are also attached to the polymer. Due to the adsorption of the gases, the amount of fluorescence emission is altered which leads to unique signals for different gases. As the fluorescence from the fluorophore is dependent on the amount and type of gases bound to the polymer, a qualitative and quantitative assessment of the gases can be made. A number of different types of polymer may be applied to the wells so that different types of gases may be detected and identified from their fingerprint. The polymer containing the fluorophore may also include a quencher, such as, colloidal gold adsorption (leading to swelling of contraction or the matrix) the fluorescence emission is changed.

A further embodiment of this invention is to pattern the FHD glass using either a high energy electron beam source or UV exposure of suitability composed glass to create a waveguide in the exposed glass, for example, by changing the refractive index. In both cases it is possible to produce opto-electronic structures including waveguides, gratings, grating couplers and filters.

The advantages of using a biochip in the above uses are that low sample volumes may be used, good signal-to-noise ratios are obtained, low diffusion times in measurement chambers and faster separations in chromatographic micro-columns can be obtained.

1. An analytical chip comprising a substrate having an array of wells arranged on the substrate for receiving a fluid and at least one waveguide positioned transversely to the wells for receiving light from said wells in response to incident light into said wells.

2. An analytical chip according to claim 1 wherein said fluid is a liquid.

3. An analytical chip according to claim 1 wherein said fluid is a gas.

4. An analytical chip according to any preceding claim wherein the wells are blind and wherein fluid does not flow through said wells.

5. An analytical chip according to any of claims 1 to 3 wherein the wells extend through said substrate to allow fluid to flow through.

6. An analytical chip according to any of claims 1 to 3 and 5 wherein the fluid is recirculated through said wells.

7. An analytical chip according to any preceding claim wherein the analytical chip is used for biological measurements and is known as a “biochip”.

8. An analytical chip according to claim 7 wherein the biochip is about 1×2.5 cm.

9. An analytical chip according to any preceding claim wherein the substrate is a silicon, silica or glass wafer about 500 μm thick with about a 10 μm thick layer of thermally grown SiO₂ on the surface.

10. An analytical chip according to any preceding claim wherein the wells are rectangular-shaped of about 50 μm wide and 50 μm deep.

11. An analytical chip according to any of claims 7 to 10 wherein the biochip comprises a measurement chamber of about 200 μm×100 μm×50 μm yielding a structure of 1 nl.

12. An analytical chip according to any preceding claim wherein the wells have one input and one output.

13. An analytical chip according to any of claims 1 to 11 wherein the wells have one input and a plurality of outputs.

14. An analytical chip according to any preceding claim wherein there is a plurality of waveguides positioned transversely to the walls.

15. An analytical chip according to any preceding claim wherein that the waveguides are about 9 μm deep.

16. An analytical chip according to any preceding claim wherein channels formed by the waveguides are arranged to improve the efficiency of light collection.

17. An analytical chip according to any preceding claim wherein, the waveguides are disposed orthogonally to the wells.

18. An analytical chip according to any preceding claim wherein the waveguides have a width varying between 9 μm and 15 μm.

19. An analytical chip according to any of claims 17 and 18 wherein the orthogonal waveguides are taper-shaped and have a starting width equal to the length of the measurement chamber.

20. An analytical chip according to claim 19 wherein the waveguides have a starting width of 200 μm or 500 μm, and have a final width of 90 μm.

21. An analytical chip according to any preceding claim wherein on the inner surface of the wells a biological molecule which can bind a ligand is attached.

22. An analytical chip according to claim 21 wherein the biological molecule has the ability to bind to a second biological molecule which contains a fluorophore group or causes a change in the optical property of the structure.

23. An analytical chip according to any of claims 1 to 20 wherein the inner surface of the wells is functionalised by a biological molecule containing a fluorophore whose optical properties are changed on binding.

24. An analytical chip according to any of claims 22 and 23 wherein the fluorophore groups are selected from those normally used in bio-analytical applications.

25. An analytical chip according to any of claims 22, 23 and 24 wherein the fluorophore groups are rhodamine and its derivatives, cyanine and its derivatives, Texas Red, proteins which contain fluorophores, natural and synthetic fluorophores, and tyrosine containing proteins.

26. A method of making an analytical chip using a flame hydrolysis deposition process comprising:
hydrolysing halides in an oxy-hydrogen flame to form a low-density oxide soot;
depositing the soot on a layer of silicon glass;
sintering the soot to form a amorphous glass;
etching the amorphous glass to form a plurality of waveguides;
depositing a further layer of amorphous glass by flame hydrolysis deposition to act as a cladding layer to the waveguide; and
then performing a further etching process on the cladding layer to form an array of flow channels.

27. An analytical chip according to claim 26 wherein the soot is deposited by using an aerosol spray.

28. An analytical chip according to any of claims 26 and 27 wherein the halides are metal halides.

29. An analytical chip according to any of claims 26 and 27 wherein the halides are SiCl₄, GeCl₄, BCl₃, and POCl₃.

30. An analytical chip according to any of claims 26 to 29 wherein the halides are in different feedlines to enable sequential deposition or co-deposition.

31. An analytical chip according to any of claims 26 to 30 wherein the soot forming the waveguide layer is sintered at 1350° C. for 2 hours forming a layer of 9 μm.

32. An analytical chip according to any of claims 26 to 31 wherein a mask is used in the second flame hydrolysis deposition layer during the etching process.

33. An analytical chip according to any of claims 26 to 32 wherein the final device is sintered at 1100° C. for 2 hours.

34. An analytical chip according to any of claims 26 to 33 wherein on the surface of the flow channels, biological molecules or fluorescently labelled biomolecules are added by injecting an immobilisation solution of saturated primer solution through the flow channel.

35. An analytical chip according to claim 34 wherein the primer solution is a functionalised silane.

36. An analytical chip according to any of claims 26 to 35 wherein the deposition procedure is repeated to provide a multi-biochip structure.

37. An analytical chip according to any of claims 26 to 35 wherein a multi-layer chip structure is obtained by forming a chip with a waveguide and flow channel layer and then using anodic bonding to bond said chip onto another chip.

38. An analytical chip according to claim 37 wherein the anodic bonding method comprises disposing a thin layer of Ti or Ni between the surfaces of two adjacent chips.

39. An analytical chip according to any of claims 26 to 35 wherein a multi-layer chip structure is obtained by using an adhesive layer (e.g. a polymer or a glass) to attach two single layer biochips.

40. Apparatus for fluorescence measurements comprising:
a light source for irradiating an analytical chip in a first direction with incident radiation; and
a light detection system for collecting emerging light from the analytical chip said emerging light being in a direction substantially in-line to said first direction.

41. Apparatus for fluorescence measurements comprising:
a light source for irradiating an analytical chip in a first direction with incident radiation; and
a light detection system for collecting emerging light from the biochip, said emerging light being in a direction substantially orthogonal to said first direction.

42. An analytical chip according to any of claims 40 and 41 wherein the analytical chip is a biochip.

43. An analytical chip according to any of claims 40, 41 and 42 wherein the light source is a HeNe laser.

44. An analytical chip according to any of claims 40 to 43 wherein the detection system is a CCD.

45. An integrated analytical chip comprising:
a light source for irradiating an analytical chip in a first direction with incident radiation; and
a light detection system for collecting emerging light from the analytical chip, said emerging light being in a direction substantially orthogonal to said first direction.

46. An integrated analytical chip comprising:
a light source for irradiating an analytical chip in a first direction with incident radiation; and
a light detection system for collecting emerging light from the analytical chip, said emerging light being in a direction substantially in-line to said first direction.

47. A point-of-care system for detecting a biological or non-biological molecule or component, said system comprising:
an analytical chip according to at least the first aspect of the invention;
a light source for irradiating the analytical chip with incident light;
a light sensor for detecting non-incident light from said chip, said non-incident light containing information about said biological or non-biological molecule or component;
signal processing means for processing said non-incident light and for extracting data representative of said biological or non-biological molecule or component and means for presenting the results of said detection.

48. A point-of-care system according to claim 47 wherein the means are displayed visually, transmitted audibly or transmitted remotely.

49. A point-of-care system according to any of claims 47 and 48 wherein the molecules are those normally required for clinical measurement e.g. DNA, RNA, proteins, enzymes, antibodies of antigens.
